(19) United States
(12) Patent Application Publication
(10) Pub. No.: US 2025/0302281 A1
(43) Pub. Date: Oct. 2, 2025

(54) ENDOSCOPE

(71) Applicant: FUJIFILM Corporation, Tokyo (JP)

(72) Inventor: Naoki SUGIURA, Kanagawa (JP)

(73) Assignee: FUJIFILM Corporation, Tokyo (JP)

(21) Appl. No.: 19/075,712

(22) Filed: Mar. 10, 2025

(30) Foreign Application Priority Data
Mar. 29, 2024 (JP) 2024-055395

(51) Int. Cl. A61B 1/00; A61B 90/00

(52) U.S. Cl. CPC A61B 1/00137; A61B 1/00096; A61B 1/00098; A61B 90/03; A61B 2090/034

(57) ABSTRACT

Provided is an endoscope in which a distal end cap can be easily attached to and detached from a distal end member and a problem in which a distal end cap is turned up during treatment can be eliminated. A protruding portion having an inner vertical surface that faces an X(+) direction side and an upper surface that faces a Z(+) direction side is provided on an end surface of a first wall portion, and a distal end cap includes flat surfaces that engage with the inner vertical surface and the upper surface.

### CROSS-REFERENCE TO RELATED APPLICATION

The present application claims priority under 35 U.S.C § 119(a) to Japanese Patent Application No. 2024-055395 filed on Mar. 29, 2024, which is hereby expressly incorporated by reference, in its entirety, into the present application.

### BACKGROUND OF THE INVENTION

### 1. Field of the Invention

The present invention relates to an endoscope, and particularly to an endoscope in which a distal end cap is mounted on a distal end member of an insertion part.

### 2. Description of the Related Art

In an endoscope, various treatment tools are introduced from a treatment tool inlet port provided in an operating part, and the treatment tool is led out from a treatment tool outlet port that is open at a distal end portion of an insertion part to the outside and used for a treatment. For example, in the duodenoscope, a treatment tool, such as a contrast tube or a guide wire, is used. In addition, in an ultrasound endoscope, a treatment tool, such as a puncture needle, is used, and in other direct-viewing endoscopes and oblique-viewing endoscopes, a treatment tool, such as forceps or a snare, is used.

In such a treatment tool, it is necessary to change a lead-out direction at the distal end portion of the insertion part in order to treat a desired position in a subject. Therefore, the distal end portion of the insertion part is provided with an elevator that changes the lead-out direction of the treatment tool. In addition, the endoscope is provided with a treatment tool elevating mechanism, and a posture of the elevator is changed between an elevated position and a fallen position by the treatment tool elevating mechanism.

Meanwhile, the distal end member constituting the distal end portion of the insertion part is made of a metal. Therefore, the distal end member is covered with the distal end cap made of a resin. The distal end cap is removed from the distal end member during washing of the endoscope performed after the treatment.

Here, JP1997-299315A (JP-H9-299315) discloses an endoscope in which an L-shaped locking protruding portion is provided on a distal end surface of a distal end member, and the locking protruding portion is fitted into a locking recessed portion formed on a distal end cover to mount the distal end cover on the distal end member.

In addition, WO2019/116637A discloses an endoscope in which a distal end cover is mounted on a distal end member by engaging a locking claw formed on the distal end cover with an engaging groove formed on the distal end member.

### SUMMARY OF THE INVENTION

In the endoscope in which the distal end cap is mounted on the distal end member, the distal end cap comes into contact with a body cavity wall or the like during the treatment of the endoscope. In a case in which the insertion part is inserted or a bendable portion is operated to be bent in a state in which the distal end cap is in contact with the body cavity wall, there is a problem in that the distal end cap (particularly, a cap distal end portion of the distal end cap) is turned up from the distal end member by receiving a force from the body cavity wall.

The above-described problem may be eliminated by increasing a fixing force (fitting force or engaging force) of a connection structure between the distal end member and the distal end cap, but, in a case in which a structure for increasing the fixing force is adopted, there is a problem in that it is difficult to easily attach and detach the distal end cap to and from the distal end member.

The present invention has been made in view of the above-described circumstances, and an object of the present invention is to provide an endoscope in which a distal end cap can be easily attached to and detached from a distal end member and a problem in which a distal end cap is turned up during treatment can be eliminated.

A first aspect relates to an endoscope comprising: a distal end member that is provided at a distal end portion of an insertion part; and a distal end cap that is mounted on the distal end member, in which, in a case in which an axial direction of the distal end member is defined as a first direction, one direction of two directions orthogonal to the first direction and orthogonal to each other is defined as a second direction, and the other direction of the two directions is defined as a third direction, the distal end member has an elevator housing portion in which an elevator is housed, and a first wall portion that is disposed in the elevator housing portion on one side in the second direction and that has an observation window and an illumination window, the distal end cap has, on one side in the third direction, a cap opening portion that exposes the observation window, the illumination window, and the elevator housing portion, the first wall portion has an engaging portion that is provided on an end surface on one side in the first direction, the engaging portion has a first engaging surface that faces the other side opposite to the one side in the second direction, and a second engaging surface that faces the one side in the third direction, and the distal end cap has an engaged portion that engages with the first engaging surface and the second engaging surface.

A second aspect relates to the endoscope according to the first aspect, in which the engaging portion is a protruding portion that protrudes from the end surface to the one side in the first direction.

A third aspect relates to the endoscope according to the second aspect, in which the protruding portion has an inner inclined surface that is connected to the first engaging surface and that is inclined toward the one side in the third direction with respect to the first engaging surface.

A fourth aspect relates to the endoscope according to the second or third aspect, in which the protruding portion has a distal end surface that is connected to the first engaging surface and the second engaging surface and that faces the one side in the first direction, an outer peripheral curved surface that is connected to the second engaging surface and the distal end surface and that faces the one side in the second direction, and a distal end inclined surface that is disposed on the other side in the third direction with respect to the distal end surface via a fillet surface and that is inclined toward both the one side in the first direction and the other side in the third direction.

A fifth aspect relates to the endoscope according to any one of the second to fourth aspects, in which the end surface of the first wall portion has, on a region on the other side in the third direction with respect to the protruding portion, an inclined wall surface that is inclined toward both the one side in the first direction and the other side in the third direction, and the distal end cap has a wall surface abutment surface that is capable of abutting on the inclined wall surface in a case in which the distal end cap is mounted on the distal end member.

A sixth aspect relates to the endoscope according to any one of the first to fifth aspects, in which the first wall portion has a cap mounting surface that is disposed on the one side in the first direction with respect to the illumination window and that faces the one side in the third direction, and the cap mounting surface is covered with the distal end cap in a case in which the distal end cap is mounted on the distal end member.

A seventh aspect relates to the endoscope according to the sixth aspect, in which the cap mounting surface is disposed on the other side in the third direction with respect to a disposition surface of the illumination window.

An eighth aspect relates to the endoscope according to the sixth or seventh aspect, in which the second engaging surface is disposed on the other side in the third direction with respect to the cap mounting surface.

A ninth aspect relates to the endoscope according to any one of the first to eighth aspects, in which the distal end member has a second wall portion that is disposed in the elevator housing portion on the other side in the second direction, and the distal end cap has a housing portion that is capable of housing a part of the second wall portion.

A tenth aspect relates to the endoscope according to the ninth aspect, in which the second wall portion has, on the one side in the first direction, a thin-wall portion that is formed to be thinner than other portions, and the housing portion is capable of housing the thin-wall portion.

An eleventh aspect relates to the endoscope according to any one of the first to tenth aspects, in which the distal end member has a base portion that is disposed in the first wall portion on the other side opposite to the one side in the first direction, a flange portion that protrudes in a radial direction is provided in an outer peripheral portion of the base portion, and the distal end cap has a cap locking portion that locks the flange portion.

A twelfth aspect relates to the endoscope according to the eleventh aspect, in which the base portion has a flat portion that is disposed on the other side in the first direction with respect to the flange portion and of which a part in a circumferential direction is formed to be flat.

According to the aspects of the present invention, it is possible to easily attach and detach the distal end cap to and from the distal end member and to eliminate the problem in which the distal end cap is turned up during the treatment.

### BRIEF DESCRIPTION OF THE DRAWINGS

FIG. 1 is an explanatory view showing an overall configuration of an endoscope.

FIG. 2 is an enlarged perspective view showing a case in which a distal end portion is viewed from a Z(+) direction side.

FIG. 3 is a perspective view showing a state in which a distal end cap is removed from a distal end member of FIG. 2.

FIG. 4 is a perspective view showing an appearance of the distal end cap.

FIG. 5 is a cross-sectional view showing the distal end cap.

FIG. 6 is a cross-sectional view showing a case in which the distal end portion is cut along a Y-Z plane.

FIG. 7 is a perspective view showing the distal end member as viewed from a Y(+) direction side.

FIG. 8 is a perspective view showing a state in which a cover shown in FIG. 7 is removed from the distal end member.

FIG. 9 is a perspective view showing the distal end member as viewed from the Y(+) direction side.

FIG. 10 is a side view showing the distal end member shown in FIG. 9 as viewed from an X(+) direction side.

FIG. 11 is a perspective view showing the distal end member as viewed from the Y(+) direction side.

FIG. 12 is a front view showing the distal end member as viewed from the Y(+) direction side.

FIG. 13 is an enlarged perspective view showing a distal end portion of a first wall portion of the distal end member.

FIG. 14 is a perspective view showing main parts of the distal end portion in which the distal end cap is mounted on the distal end member.

FIG. 15 is a perspective view showing a mounting portion of the distal end cap as viewed from a Y(−) direction side.

FIG. 16 is a cross-sectional view showing another inclined surface provided on the distal end cap.

FIG. 17 is a perspective view showing a housing portion of the distal end cap as viewed from the Y(−) direction side.

### DESCRIPTION OF THE PREFERRED EMBODIMENTS

Hereinafter, an endoscope according to an embodiment of the present invention will be described with reference to the accompanying drawings.

### Overall Configuration of Endoscope 1

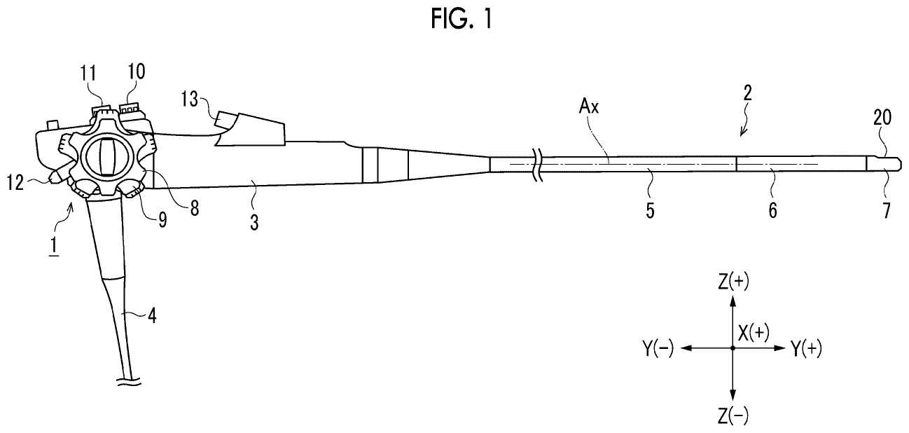

FIG. 1 is an overall configuration view showing an endoscope 1 according to the present embodiment.

As shown in FIG. 1, the endoscope 1 comprises an insertion part 2 that is inserted into a subject, and an operating part 3 that is connected to a base end portion of the insertion part 2 and is operated by an operator. It should be noted that, in the embodiment, a side-viewing endoscope used as a duodenoscope is described as an example.

Hereinafter, in a case of describing a configuration of each part of the endoscope 1, a three-dimensional orthogonal coordinate system of X, Y, and Z will be used for convenience of description. A Z direction in the drawings is an up-down direction, a Z(+) direction side indicates an up direction, and a Z(−) direction side indicates a down direction. In addition, an X direction in the drawings is a direction perpendicular to the Z direction, an X(+) direction side indicates a right direction, and an X(−) direction side indicates a left direction. In addition, a Y direction in the drawings is a direction perpendicular to both the Z direction and the X direction, a Y(+) direction side indicates a distal end side direction, and a Y(−) direction side indicates a base end side direction. It should be noted that, in addition, each of the above-described directions refers to a direction in a case in which the operator views the insertion part 2 from an upper side in a case in which the operator grips the operating part 3 of the endoscope 1.

In addition, the Y direction, the X direction, and the Z direction correspond to a first direction, a second direction, and a third direction according to the embodiment of the present invention, respectively. In addition, the Y(+) direction and the Y(−) direction correspond to a direction on one side of the first direction and a direction on the other side of the first direction according to the embodiment of the present invention, respectively. In addition, the X(+) direction and the X(−) direction correspond to a direction on the other side of the second direction and a direction on one side of the second direction according to the embodiment of the present invention, respectively. In addition, the Z(+) direction and the Z(−) direction correspond to a direction on one side of the third direction of the present invention and a direction on the other side of the third direction according to the embodiment of the present invention, respectively.

### Insertion Part 2

The insertion part 2 of the endoscope 1 is inserted into the subject through an oral cavity and is further inserted into a duodenum from an esophagus via a stomach. Accordingly, a procedure such as an examination or a treatment of the duodenum is performed by using a treatment tool inserted into the insertion part 2.

Examples of the treatment tool include biopsy forceps having a cup that can collect a biological tissue at a distal end portion thereof, an endoscopic sphincterotomy (EST) knife, a contrast tube, and a guide wire.

The insertion part 2 has a longitudinal axis Ax, and has a soft portion 5, a bendable portion 6, and a distal end portion 7 in order from a base end side to a distal end side. It should be noted that a longitudinal axis Ax direction and the Y direction are the same direction.

The soft portion 5 occupies most of the insertion part 2 from the base end side, and is formed by, for example, a metal spiral tube and a net (not shown), and is further formed by a resin tube covering the outer periphery of the metal spiral tube and the net. The soft portion 5 has flexibility that allows the soft portion 5 to be bent in any direction, and is bent along an insertion path into a body cavity in a case in which the insertion part 2 is inserted into the body cavity.

The bendable portion 6 has a tubular body in which a plurality of angle rings are connected to each other in a swingable manner, and is formed by covering an outer periphery of the tubular body with a mesh body woven with a metal wire and further covering the outer periphery of the mesh body with angle rubber made of rubber. In addition, a plurality of wires extend from the angle knobs 8 and 9 of the operating part 3 to the bendable portion 6, and distal end portions of these wires are fixed to the distal end portion of the tubular body. Accordingly, the bendable portion 6 is bent in the up-down direction (Z direction) and the left-right direction (X direction) in response to a rotational movement operation of the angle knobs 8 and 9.

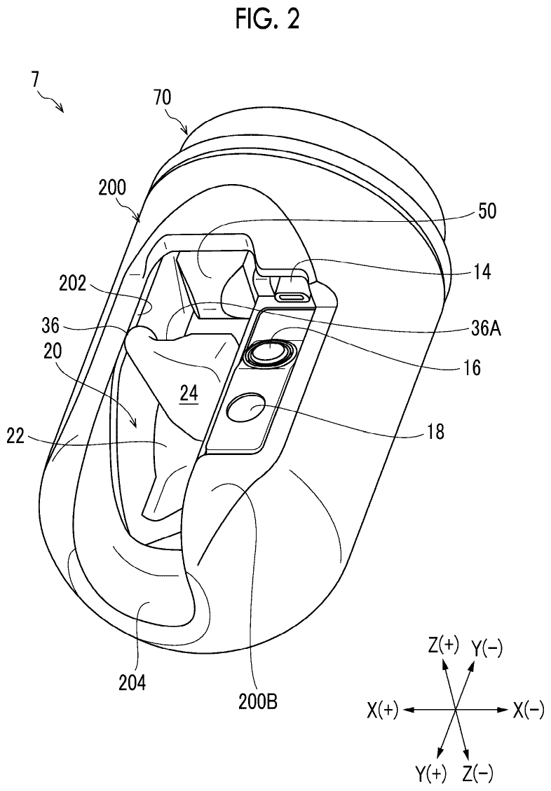

Although the distal end portion 7 will be described in detail below with reference to FIG. 2 and the following drawings, the distal end portion 7 has the distal end member 20 and a distal end cap 200 (see FIG. 2) that is attachably and detachably mounted on the distal end member 20.

The distal end member 20 is provided at the distal end portion 7, and is made of a metal material having corrosion resistance. An axial direction of the distal end member 20 is the same as the longitudinal axis Ax direction and the Y direction. The distal end cap 200 is made of an elastic material, for example, silicone rubber. The distal end member 20 and the distal end cap 200 are examples of a distal end member and a distal end cap according to the embodiment of the present invention, respectively.

### Operating Part 3

As shown in FIG. 1, the operating part 3 includes angle knobs 8 and 9 for bending the bendable portion 6, an air/water supply button 10, a suction button 11, an elevating operation lever 12, and a treatment tool inlet port 13.

The air/water supply button 10 is an operation button for performing air/water supply from an air/water supply nozzle 14 (see FIG. 2) of the distal end portion 7 toward an observation window 16, an illumination window 18, and the like via an air/water supply channel (not shown) inserted into the insertion part 2.

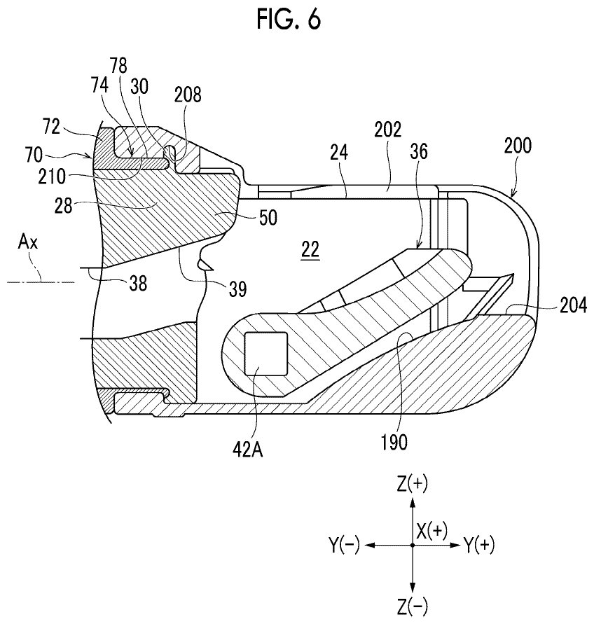

The suction button 11 is an operation button for suctioning a body fluid and the like via a treatment tool insertion channel 38 (see FIG. 6) inserted into the insertion part 2.

The elevating operation lever 12 is a lever for performing an operation of elevating and lowering an elevator 36 (see FIG. 2) provided at the distal end portion 7. The elevating operation lever 12 is one of constituent members of a treatment tool elevating mechanism.

The treatment tool inlet port 13 communicates with the treatment tool insertion channel 38, and is an opening through which the operator introduces the treatment tool or the like. The treatment tool introduced from the treatment tool inlet port 13 is guided to the distal end portion 7 through the treatment tool insertion channel 38.

A universal cord 4 is connected to the operating part 3, and the universal cord 4 is connected to an image processing device and a light source device (not shown). A monitor device (not shown) is connected to the image processing device via a cable. The overall structure of the endoscope 1 is as described above. Hereinafter, a structure of the distal end portion 7 will be described.

### Distal End Portion 7

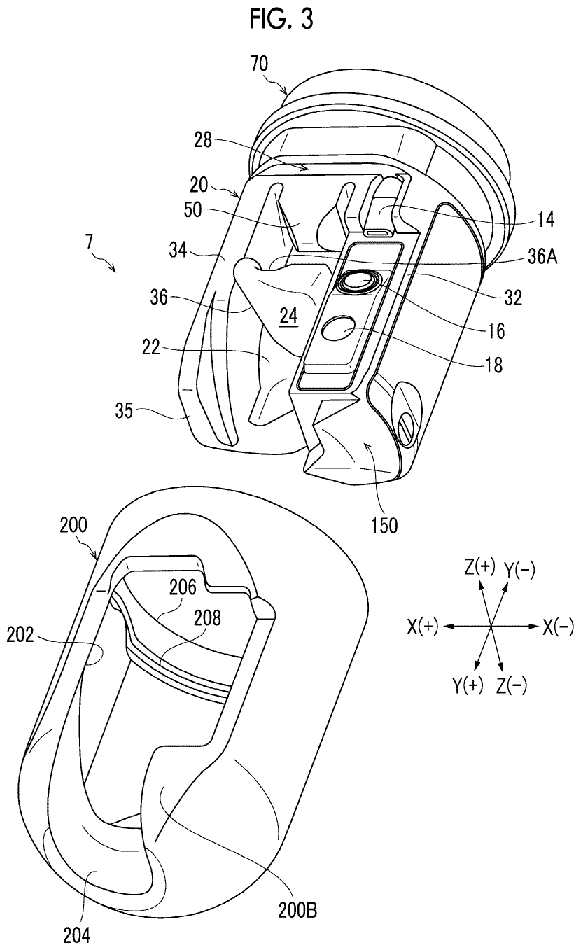

FIG. 2 is an enlarged perspective view showing a case in which the distal end portion 7 is viewed from the Z(+) direction side. FIG. 2 shows a state in which the distal end cap 200 is mounted on the distal end member 20. In addition, FIG. 3 is a perspective view showing a state in which the distal end cap 200 is removed from the distal end member 20 of FIG. 2.

As shown in FIG. 2, in a state in which the distal end cap 200 is mounted on the distal end member 20, the distal end cap 200 has an upper opening 202 that communicates with an opening 24 on the Z(+) direction side of an elevator housing portion 22 to be described later.

Subsequently, a configuration of the distal end cap 200 will be briefly described. A detailed configuration of the distal end cap 200 will be described below.

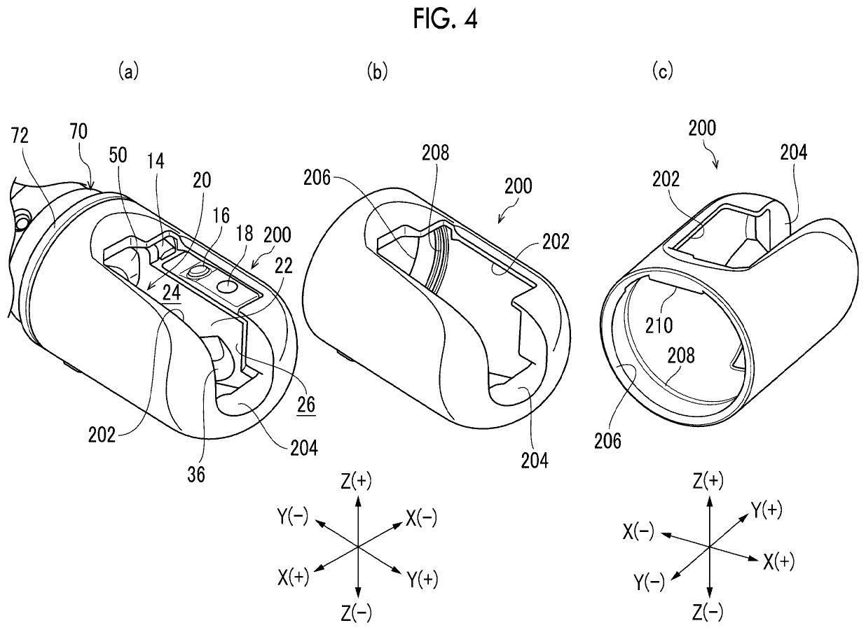

FIG. 4 is a perspective view showing an appearance of the distal end cap 200. In FIG. 4, (a) is a perspective view showing a state in which the distal end cap 200 is mounted on the distal end member 20 as viewed from the Y(+) direction side, (b) is a perspective view showing the distal end cap 200 as viewed from the Y(+) direction side, and (c) is a perspective view showing the distal end cap 200 as viewed from the Y(−) direction side.

As shown in FIG. 4, the distal end cap 200 is formed in a substantially tubular shape to house the distal end member 20. In addition, the distal end cap 200 has an upper opening 202 that communicates with the opening 24 of the elevator housing portion 22, a distal end portion opening 204 that communicates with a distal end opening 26 of the elevator housing portion 22 on the Y(+) direction side, and a base end portion opening 206 for mounting the distal end cap 200 on the distal end member 20.

The upper opening 202 is provided on the Z(+) direction side, and is formed to have a size at which the distal end portion of the air/water supply nozzle 14, the observation window 16, the illumination window 18, and a guide wire fixing portion 50 described later are exposed in addition to the elevator housing portion 22 (opening 24). In addition, the distal end portion opening 204 is installed consecutively to the upper opening 202, and is formed as an opening for air venting for preventing a body cavity wall from being suctioned to the upper opening 202 during a suction operation of the endoscope 1. In addition, the base end portion opening 206 is an opening for inserting the distal end member 20. It should be noted that the upper opening 202 corresponds to a cap opening portion according to the embodiment of the present invention.

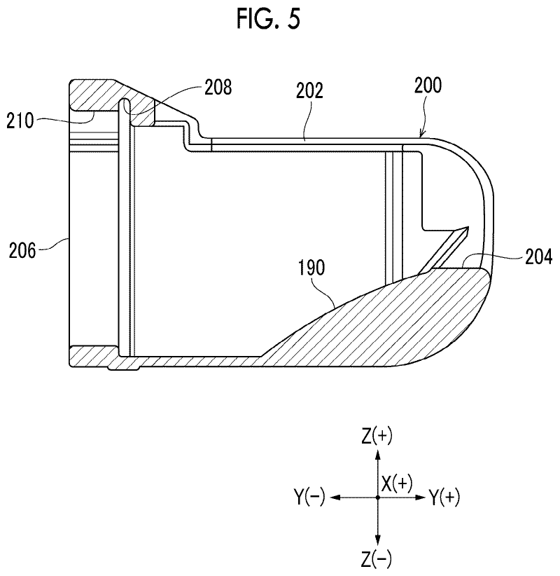

FIG. 5 is a cross-sectional view showing the distal end cap 200 in a case in which the distal end cap 200 is cut along a Z-Y plane. FIG. 6 is a cross-sectional view showing a case in which the distal end portion 7 shown in FIG. 2 is cut along the Y-Z plane.

As shown in FIGS. 5 and 6, the distal end cap 200 has an engaging groove 208 and a flat portion 210 on an inner peripheral surface on a base end side. The engaging groove 208 is provided over the entire inner peripheral surface of the distal end cap 200. The flat portion 210 is a surface of the inner peripheral surface of the distal end cap 200 on the Z(+) direction side, and is provided on a surface on the base end side (Y(−) direction side) with respect to the engaging groove 208. In addition, the flat portion 210 has, for example, a surface substantially parallel to an X-Y plane.

As shown in FIG. 6, the engaging groove 208 is locked to a flange portion 30 that protrudes on an outer peripheral surface of a base portion 28 of the distal end member 20. Therefore, the distal end cap 200 is mounted on the distal end member 20. In addition, in a case in which the distal end cap 200 is mounted on the distal end member 20, the flat portion 210 abuts on the flat portion 78 provided in an insulating ring 70 described later. It should be noted that the flange portion 30 and the engaging groove 208 are examples of a flange portion and a cap locking portion according to the embodiment of the present invention, respectively.

Subsequently, a configuration of the distal end member 20 will be briefly described. A detailed configuration of the distal end member 20 will be described below.

As shown in FIG. 3, the distal end member 20 has a first wall portion 32 and a second wall portion 34 that face each other in the X direction. The first wall portion 32 is disposed on the X(−) direction side, and the second wall portion 34 is disposed on the X(+) direction side.

In addition, the first wall portion 32 and the second wall portion 34 are installed consecutively to a base portion 28 of the distal end member 20 at the respective base ends thereof, and extend from the base portion 28 to the Y(+) direction side. It should be noted that the base portion 28, the first wall portion 32, and the second wall portion 34 are examples of a base portion, a first wall portion, and a second wall portion according to the embodiment of the present invention, respectively.

Further, the elevator housing portion 22 for housing the elevator 36 is provided between the first wall portion 32 and the second wall portion 34 in the X direction. The elevator housing portion 22 is provided adjacent to the first wall portion 32 and the second wall portion 34, and is formed as a slit-shaped space along the Y direction. It should be noted that the elevator housing portion 22 is an example of an elevator housing portion according to the embodiment of the present invention.

A treatment tool outlet port 39 (see FIG. 6) is provided in the distal end member 20. The distal end side of the treatment tool insertion channel 38 communicates with the treatment tool outlet port 39. The treatment tool insertion channel 38 is inserted into the insertion part 2 (see FIG. 1), and the base end side of the treatment tool insertion channel 38 is connected to the treatment tool inlet port 13 of the operating part 3.

Therefore, the treatment tool introduced from the treatment tool inlet port 13 is guided to the elevator housing portion 22 through the treatment tool insertion channel 38 and the treatment tool outlet port 39. It should be noted that the treatment tool guided to the elevator housing portion 22 is changed in a lead-out direction by the elevator 36.

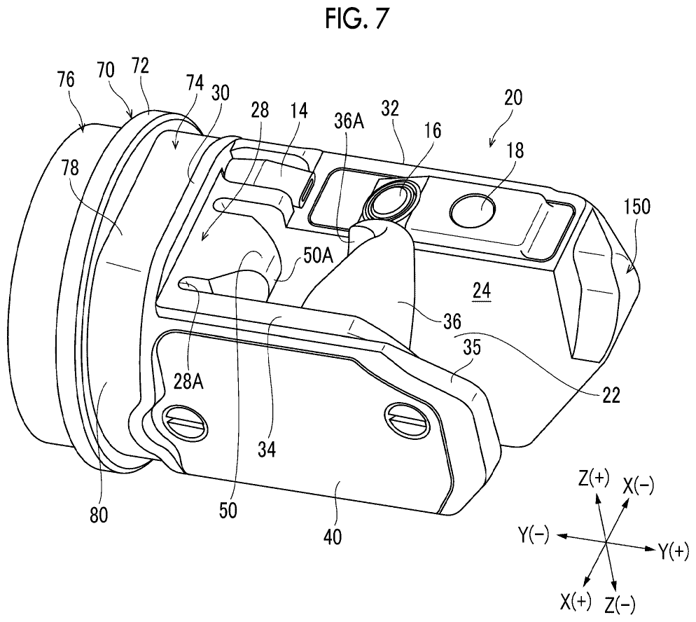

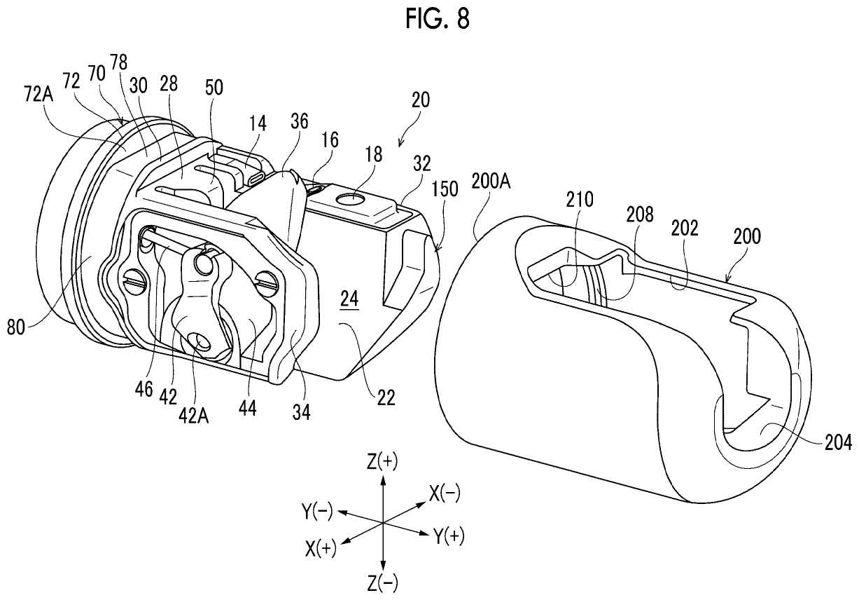

FIG. 7 is a perspective view showing the distal end member 20 as viewed from the Y(+) direction side. FIG. 8 is a perspective view showing a state in which a cover 40 shown in FIG. 7 is removed from the second wall portion 34, and in FIG. 8, the distal end cap 200 is also shown.

### First Wall Portion 32

As shown in FIGS. 7 and 8, the observation window 16 and the illumination window 18 are provided on the first wall portion 32. In addition, an imaging unit and an illumination unit are provided in the first wall portion 32.

The imaging unit has an imaging optical system disposed inside the observation window 16 and a complementary-metal-oxide semiconductor (CMOS) type or charge-coupled device (CCD) type imaging element. The imaging element is connected to an image processing device via a signal cable inserted into the insertion part 2. An imaging signal of a subject image obtained by the imaging unit is input to the image processing device through the signal cable, and is displayed as the subject image on the monitor connected to the image processing device through the cable.

The illumination unit includes an illumination lens installed inside the illumination window 18 and a light guide disposed such that a distal end thereof faces the illumination lens. The light guide is inserted into the insertion part 2, and a base end portion thereof is connected to the light source device. As a result, irradiation light from the light source device is transmitted through the light guide and is emitted from the illumination window 18.

### Second Wall Portion 34

As shown in FIG. 8, a lever housing chamber 44 that has a recessed shape and that houses a lever 42 is provided in the second wall portion 34. Among two wall surfaces of the second wall portion 34 facing each other in the X direction, the lever housing chamber 44 is provided on a wall surface on a side opposite to a wall surface that faces the elevator housing portion 22.

The lever 42 in the lever housing chamber 44 is provided with a rotation shaft 42A at one end portion thereof. The rotation shaft 42A penetrates the second wall portion 34 in the X direction, and is connected to the elevator 36. Further, the other end portion of the lever 42 is connected to an operating wire 46. The operating wire 46 is inserted into the insertion part 2 (see FIG. 1), and is connected to the elevating operation lever 12 of the operating part 3.

Therefore, the operator operates the elevating operation lever 12 to push or pull the operating wire 46, and the lever 42 swings in conjunction with the push-pull operation. Accordingly, the elevator 36 swings using the rotation shaft 42A as a swing shaft, and a position of the elevator 36 is changed between an elevated position and a fallen position. Here, the treatment tool elevating mechanism is formed by the lever 42 having the rotation shaft 42A, the operating wire 46, and the elevating operation lever 12. It should be noted that the lever housing chamber 44 is sealed by the cover 40 shown in FIG. 7.

### Elevator 36

As shown in FIGS. 7 and 8, the elevator 36 is provided in the distal end member 20, and is housed in the elevator housing portion 22. In addition, the elevator 36 is attached to the second wall portion 34 swingably via the rotation shaft 42A. The elevator 36 is driven by the treatment tool elevating mechanism to perform an elevating operation and a falling operation.

The elevator 36 has a guide surface 36A (see FIG. 2 and the like). The guide surface 36A is formed on a surface that faces the Z(+) direction side in a case in which the elevator 36 is located at the fallen position. The guide surface 36A is a surface for guiding the treatment tool, and is formed in a substantially U-shape that is recessed inward from the base end side that is a swing end of the elevator 36 to the distal end side.

In a case in which the elevator 36 is elevated by the treatment tool elevating mechanism, the treatment tool guided to the elevator housing portion 22 is guided by the guide surface 36A of the elevator 36 in a direction toward the opening 24 of the elevator housing portion 22 and is led out from the upper opening 202 of the distal end cap 200 to the outside. In addition, the lead-out direction of the treatment tool can be changed by adjusting an elevating angle of the elevator 36 by the treatment tool elevating mechanism.

### Guide Wire Fixing Portion 50

As shown in FIG. 7, a guide wire fixing portion 50 is provided at the base portion 28. The guide wire fixing portion 50 protrudes from a distal end wall portion 28A of the base portion 28 toward the elevator housing portion 22 on the Y(+) direction side.

The guide wire fixing portion 50 is formed as a tapered protruding portion in the Y(+) direction. In other words, the guide wire fixing portion 50 is formed to have a substantially trapezoidal cubic shape with the distal end wall portion 28A as a bottom surface.

In a case in which the elevator 36 is elevated to the maximum elevated position, a guide wire (not shown) guided from the treatment tool insertion channel 38 to the elevator housing portion 22 is interposed and fixed between the protruding distal end portion 50A of the guide wire fixing portion 50 and the guide surface 36A having a recessed shape of the elevator 36. It should be noted that the guide wire fixing portion 50 is installed adjacent to the air/water supply nozzle 14 in the X direction.

### Insulating Ring 70

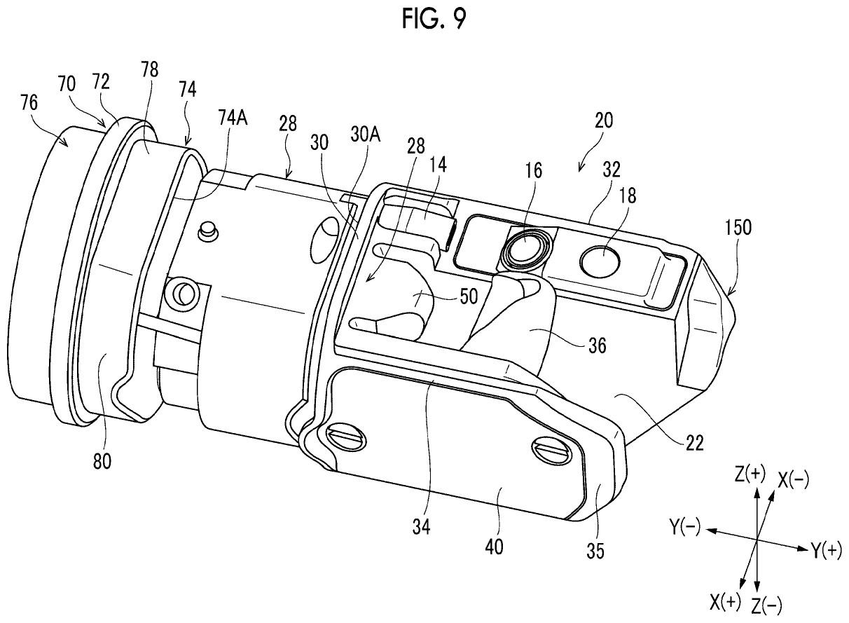

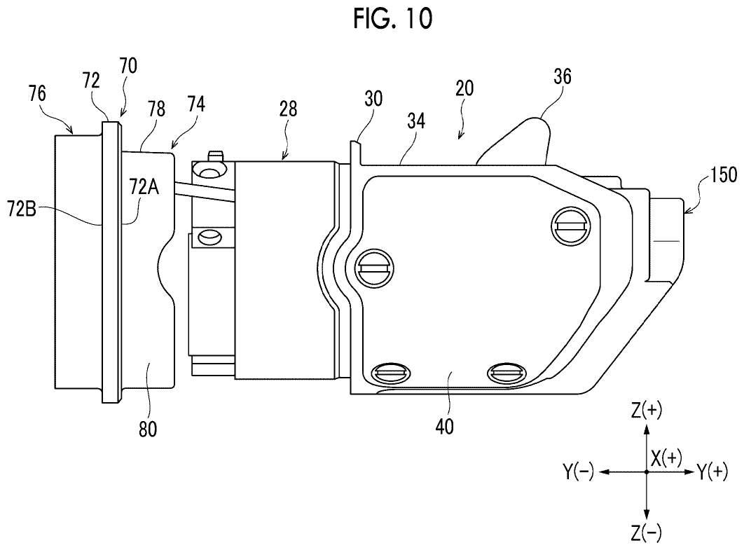

FIG. 9 is a perspective view showing the distal end member 20 as viewed from the Y(+) direction side. FIG. 10 is a side view showing the distal end member 20 shown in FIG. 9 as viewed from the X(+) direction side. It should be noted that FIGS. 9 and 10 show a state in which the insulating ring 70 is separated from the distal end member 20 to the Y(−) direction side.

As shown in FIGS. 9 and 10, the base portion 28 of the distal end member 20 is formed in a substantially cylindrical shape along the Y direction, and an insulating ring 70 having an annular shape is mounted on an outer peripheral portion thereof. The insulating ring 70 is made of an insulating material, such as plastic or ceramic.

The insulating ring 70 has a flange portion 72 that protrudes outward in the radial direction along an outer peripheral surface thereof, a cap mounting portion 74 that is disposed on the distal end side with respect to the flange portion 72, and a bendable portion mounting portion 76 that is disposed on the base end side with respect to the flange portion 72.

The cap mounting portion 74 has a flat portion 78 and an arc portion 80 on an outer peripheral surface. The flat portion 78 is provided on a surface of the outer peripheral surface on the Z(+) side, and is formed at a position (Z(−) side position) lower than the flange portion 30 of the base portion 28. This flat portion 78 has, for example, a surface substantially parallel to an X-Y plane.

In addition, the cap mounting portion 74 is formed such that an inner diameter thereof is larger than an outer diameter (diameter) of the base portion 28. The cap mounting portion 74 is externally mounted on the base portion 28 from the base end side of the base portion 28 to the distal end side, and a base end 74A of the cap mounting portion 74 abuts on a base end side surface 30A of the flange portion 30 of the base portion 28.

As a result, the cap mounting portion 74 is mounted on the base portion 28, and the insulating ring 70 is mounted on the distal end member 20. In this case, the flat portion 78 and the arc portion 80 of the insulating ring 70 are disposed on the base end side of the flange portion 30 (see FIGS. 7 and 8).

In a state shown in FIG. 8 in which the insulating ring 70 is mounted on the distal end member 20, the distal end cap 200 is mounted on the distal end member 20. In this case, the distal end cap 200 is externally mounted on the distal end member 20 from the distal end side of the distal end member 20 to the base end side, and the base end 200A of the distal end cap 200 abuts on the distal end side surface 72A of the flange portion 72 of the insulating ring 70.

Therefore, as shown in FIG. 6, the engaging groove 208 of the distal end cap 200 engages with the flange portion 30 of the distal end member 20, and the flat portion 210 of the distal end cap 200 abuts on the flat portion 78 of the insulating ring 70. As a result, the distal end cap 200 is mounted on the distal end member 20, and the distal end member 20 made of a metal is covered with the distal end cap 200 having the insulating property and the insulating ring 70.

With such a configuration, with the endoscope 1 according to the present embodiment, the insulating property of the distal end portion 7 can be ensured by the distal end cap 200 and the insulating ring 70. Further, the engagement of the flange portion 30 and the engaging groove 208 can prevent the distal end cap 200 from coming off the distal end member 20 to the Y(+) direction side. In addition, since the flat portion 78 and the flat portion 210 abut on each other, it is possible to prevent the distal end cap 200 from being displaced with respect to the distal end member 20 in a direction around the longitudinal axis Ax.

It should be noted that the base end portion of the distal end cap 200 and the flange portion 72 of the insulating ring 70 have substantially the same outer diameter (diameter). Accordingly, in a case in which the distal end cap 200 is mounted on the distal end member 20, the distal end cap 200 and the insulating ring 70 are connected to each other without a step difference in the respective outer peripheral surfaces.

In addition, in a case in which the engaging groove 208 is not completely engaged with the flange portion 30 and the distal end cap 200 is incompletely mounted on the distal end member 20, for example, in a case in which the distal end cap 200 is mounted in a state in which the base end 200A of the distal end cap 200 abuts on the flange portion 30, a part of the distal end cap 200 (for example, a part located on the base end side of the air/water supply nozzle 14) enters an observation visual field of the imaging unit. In this case, a part of the distal end cap 200 is reflected as black on an observation screen displayed on the monitor device. As a result, the operator can be notified of the incomplete mounting.

As shown in FIGS. 9 and 10, the bendable portion mounting portion 76 is formed in a cylindrical shape. The bendable portion mounting portion 76 is a portion on which a distal end portion of the angle rubber of the bendable portion 6 is mounted. The distal end portion of the angle rubber of the bendable portion 6 is mounted on the bendable portion mounting portion 76, and the distal end of the angle rubber abuts on the base end side surface 72B of the flange portion 72. As a result, the angle rubber can be mounted on the insulating ring 70. It should be noted that the angle rubber is fixed to the insulating ring 70 by winding a thread (not shown) around the distal end portion thereof. The thread is fixed to the angle rubber with an adhesive (not shown).

As in the endoscope 1 according to the present embodiment in which the distal end cap 200 is mounted on the distal end member 20, there is a problem in that the distal end cap 200 is turned up from the distal end member 20 because the distal end cap 200 receives a force from the body cavity wall.

In particular, in a case in which a cap distal end portion 200B (see FIG. 2 and the like) located on the distal end side of the first wall portion 32 among the distal end portions of the distal end cap 200 is turned up, the liquid may enter the inside of the first wall portion 32 from the turned-up portion, and thus it is necessary to prevent the turning-up of the cap distal end portion 200B.

The above-described problem may be eliminated by increasing a fixing force (fitting force or engaging force) of a connection structure between the distal end member 20 and the distal end cap 200, but, in a case in which a structure for increasing the fixing force is adopted, there is a problem in that it is difficult to easily attach and detach the distal end cap 200 to and from the distal end member 20.

Therefore, the endoscope 1 according to the present embodiment has the following configuration in order to facilitate attachment and detachment of the distal end cap 200 to and from the distal end member 20 and to eliminate the problem in which the distal end cap 200 is turned up during the treatment. Hereinafter, the description thereof will be made in detail.

First, the distal end member 20 will be described.

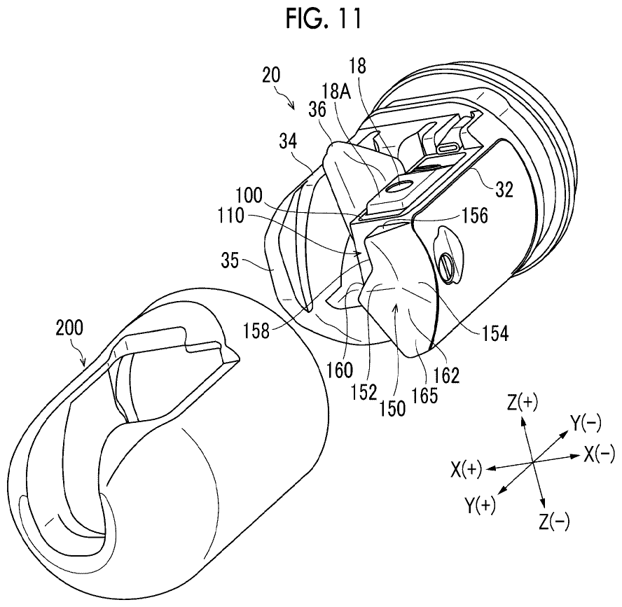

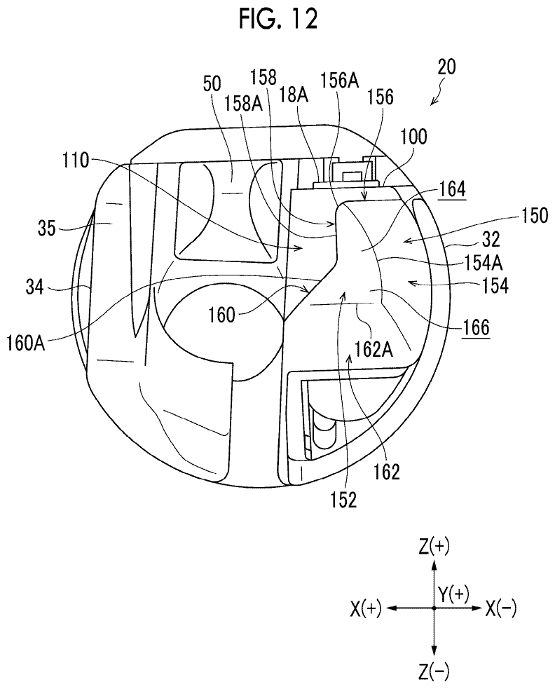

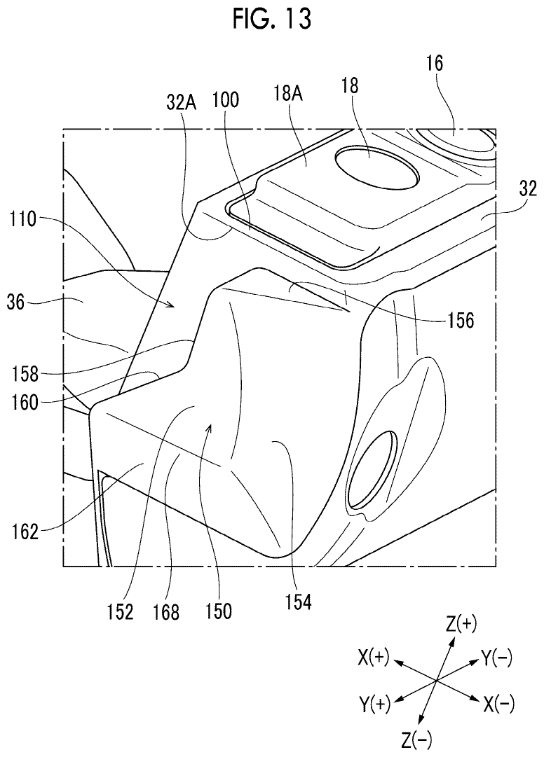

FIG. 11 is a perspective view showing the distal end member 20 as viewed from the Y(+) direction side. FIG. 11 also shows the distal end cap 200. FIG. 12 is a front view showing the distal end member 20 as viewed from the Y(+) direction side. FIG. 13 is a enlarged perspective view showing the distal end portion of the first wall portion 32 of the distal end member 20. It should be noted that, in the drawings from FIG. 11 onwards, in order to avoid complication of reference numerals, reference numerals necessary for description will be added, and unnecessary reference numerals will be omitted.

### Cap Mounting Surface 100

As shown in FIGS. 11 to 13, the first wall portion 32 has a cap mounting surface 100 on an upper surface (surface on the Z(+) direction side) on the Y(+) direction side. This cap mounting surface 100 is disposed on the Y(+) direction side with respect to the illumination window 18.

In addition, the cap mounting surface 100 is formed by a surface parallel to the X-Y plane and faces the Z(+) direction side. It should be noted that the cap mounting surface 100 need only be formed by at least a surface that faces the Z(+) direction side. That is, the cap mounting surface 100 need only be formed by a surface including a normal component toward the Z(+) direction side, and may be, for example, a surface substantially parallel to the X-Y plane (surface slightly inclined with respect to the X-Y plane).

Further, the cap mounting surface 100 is disposed on the Z(−) direction side with respect to a disposition surface 18A of the illumination window 18. In a case in which the distal end cap 200 is mounted on the distal end member 20, the cap mounting surface 100 is covered with the cap distal end portion 200B (see FIG. 2 and the like) of the distal end cap 200. It should be noted that the cap mounting surface 100 is an example of a cap mounting surface according to the embodiment of the present invention.

### End Surface 110

The first wall portion 32 has an end surface 110 on the Y(+) direction side. The end surface 110 is installed consecutively to the cap mounting surface 100 via a distal end corner portion 32A of the first wall portion 32.

In addition, the end surface 110 is formed by a surface parallel to the X-Z plane and faces the Y(+) direction side. It should be noted that the end surface 110 need only be formed by at least a surface that faces the Y(+) direction side. That is, the end surface 110 need only be formed by a surface including a normal component toward the Y(+) direction side, and may be, for example, a surface substantially parallel to the X-Z plane (surface slightly inclined with respect to the X-Z plane). It should be noted that the end surface 110 is an example of an end surface on one side in the first direction according to the embodiment of the present invention.

### Protruding Portion 150

The first wall portion 32 comprises a protruding portion 150 on the end surface 110. The protruding portion 150 is formed as a protruding portion that protrudes to the Y(+) direction side with the end surface 110 as a bottom surface. It should be noted that the protruding portion 150 is an example of an engaging portion according to the embodiment of the present invention, and is an example of a protruding portion. Hereinafter, a configuration of the protruding portion 150 will be described in detail.

The protruding portion 150 has a plurality of surfaces. That is, the protruding portion 150 has a distal end surface 152, an outer peripheral curved surface 154, an upper surface 156, an inner vertical surface 158, an inner inclined surface 160, and a distal end inclined surface 162.

### Distal End Surface 152

The distal end surface 152 is a surface located on the most Y(+) direction side, among the plurality of surfaces of the protruding portion 150. The distal end surface 152 is connected to the inner vertical surface 158 and the upper surface 156, which will be described later.

The distal end surface 152 is formed by a surface parallel to the X-Z plane, and faces the Y(+) direction side. It should be noted that the distal end surface 152 need only be formed by at least a surface that faces the Y(+) direction side. That is, the distal end surface 152 need only be formed by a surface including a normal component toward the Y(+) direction side, and may be, for example, a surface substantially parallel to the X-Z plane (surface slightly inclined with respect to the X-Z plane). It should be noted that the distal end surface 152 is an example of a distal end surface according to the embodiment of the present invention.

As shown in FIG. 12, in a case in which the distal end surface 152 is viewed from the Y(+) direction side, the distal end surface 152 is a surface defined by a ridge line 154A of the outer peripheral curved surface 154, a ridge line 156A of the upper surface 156, a ridge line 158A of the inner vertical surface 158, a ridge line 160A of the inner inclined surface 160, and a ridge line 162A of the distal end inclined surface 162.

The ridge line 154A is formed in an arc shape that bulges toward the X(−) direction side, and the ridge line 156A is formed in a linear shape along the X direction. The ridge line 158A is formed in a linear shape along the Z direction, and the ridge line 160A is formed in a linear shape inclined to both the Z(−) direction side and the X(+) direction side. The ridge line 162A is formed in a linear shape along the X direction.

The plurality of ridge lines form the distal end surface 152, and the distal end surface 152 has a substantially triangular surface 164 formed by ridge lines including three ridge lines 154A, 156A, and 158A, and a substantially trapezoidal surface 166 formed by ridge lines including three ridge lines 160A, 162A, and 154A. The distal end surface 152 is formed by these two surfaces 164 and 166.

### Outer Peripheral Curved Surface 154

The outer peripheral curved surface 154 is connected to the upper surface 156 and the distal end surface 152 and is formed by a surface extending from a position on the X(−) direction side to the Y(+) direction side of the end surface 110.

The outer peripheral curved surface 154 faces the X(−) direction side, and has a curved surface that bulges in an arc shape toward the X(31 ) direction side. It should be noted that the outer peripheral curved surface 154 need only be formed by at least a curved surface that faces the X(−) direction side. That is, the outer peripheral curved surface 154 need only be formed by a curved surface including a normal component toward the X(−) direction side, a normal component toward the Z(+) direction side, and a normal component toward the Z(−) direction side. It should be noted that the outer peripheral curved surface 154 is an example of an outer peripheral curved surface according to the embodiment of the present invention.

### Upper Surface 156

The upper surface 156 is formed by a surface extending from a position on the Z(+) direction side of the end surface 110 to the Y(+) direction side. The upper surface 156 is disposed on the Z(−) direction side with respect to the cap mounting surface 100.

The upper surface 156 is formed by a surface parallel to the X-Y plane and faces the Z(+) direction side. It should be noted that the upper surface 156 need only be formed by at least a surface that faces the Z(+) direction side. That is, the upper surface 156 need only be formed by a surface including a normal component toward the Z(+) direction side, and may be, for example, a surface substantially parallel to the X-Y plane (surface slightly inclined with respect to the X-Y plane). In addition, the upper surface 156 is formed as a substantially triangular surface as viewed from the Z(+) direction side. It should be noted that the upper surface 156 is an example of a second engaging surface according to the embodiment of the present invention.

### Inner Vertical Surface 158

The inner vertical surface 158 is formed by a plane extending from a position of the end surface 110 on the X(+) direction side to the Y(+) direction side.

The inner vertical surface 158 is formed by a surface parallel to the Y-Z plane and faces the X(+) direction side. It should be noted that the inner vertical surface 158 need only be formed by at least a surface that faces the X(+) direction side. That is, the inner vertical surface 158 need only be formed by a surface including a normal component toward the X(+) direction side, and may be, for example, a surface substantially parallel to the Y-Z plane (surface slightly inclined with respect to the Y-Z plane).

In addition, as viewed from the Y(+) direction side, the inner vertical surface 158 is disposed on the X(+) direction side of the outer peripheral curved surface 154 with the distal end surface 152 interposed therebetween. In addition, the inner vertical surface 158 is formed as a substantially rectangular surface as viewed from the X(+) direction side. It should be noted that the inner vertical surface 158 is an example of a first engaging surface according to the embodiment of the present invention.

### Inner Inclined Surface 160

The inner inclined surface 160 is formed by a surface extending from a position of the end surface 110 on the X(+) direction side to the Y(+) direction side.

The inner inclined surface 160 is connected to the inner vertical surface 158, and faces the Z(+) direction side with respect to the inner vertical surface 158. It should be noted that the inner inclined surface 160 need only be formed by a surface that faces at least the Z(+) direction side with respect to the inner vertical surface 158. That is, the inner inclined surface 160 need only be formed by a surface including a normal component toward the Z(+) direction side and a normal component toward the X(+) direction side.

In addition, as viewed from the Y(+) direction side, the inner inclined surface 160 is disposed on the X(+) direction side of the outer peripheral curved surface 154 with the distal end surface 152 interposed therebetween. In addition, as viewed from the X(+) direction side, the inner inclined surface 160 is formed as a substantially rectangular surface. It should be noted that the inner inclined surface 160 is an example of an inner inclined surface according to the embodiment of the present invention.

### Distal End Inclined Surface 162

The distal end inclined surface 162 is disposed on the Z(−) direction side with respect to the distal end surface 152 with a fillet surface 168 (see FIG. 13) interposed therebetween.

The distal end inclined surface 162 is a surface that faces both the Y(+) direction side and the Z(−) direction side. It should be noted that the distal end inclined surface 162 need only be formed by at least a surface that faces both the Y(+) direction side and the Z(−) direction side. That is, the distal end inclined surface 162 need only be formed by a surface including a normal component toward the Y(+) direction side and a normal component toward the Z(−) direction side.

In addition, as viewed from the Z(−) direction side, the distal end inclined surface 162 is formed as a substantially rectangular surface. It should be noted that the distal end inclined surface 162 is an example of a distal end inclined surface according to the embodiment of the present invention.

As shown in FIG. 11, the first wall portion 32 has a wall surface abutment surface 165 in a region of the end surface 110 of the first wall portion 32 on the Z(−) direction side with respect to the protruding portion 150.

The wall surface abutment surface 165 is a surface that faces both the Y(+) direction side and the Z(−) direction side, and faces the Z(−) direction side with respect to the distal end inclined surface 162. It should be noted that the wall surface abutment surface 165 need only be formed by at least a surface that faces both the Y(+) direction side and the Z(−) direction side. That is, the wall surface abutment surface 165 need only be formed by a surface including a normal component toward the Y(+) direction side and a normal component toward the Z(−) direction side. The protruding portion 150 is formed as described above.

Subsequently, the distal end cap 200 will be described.

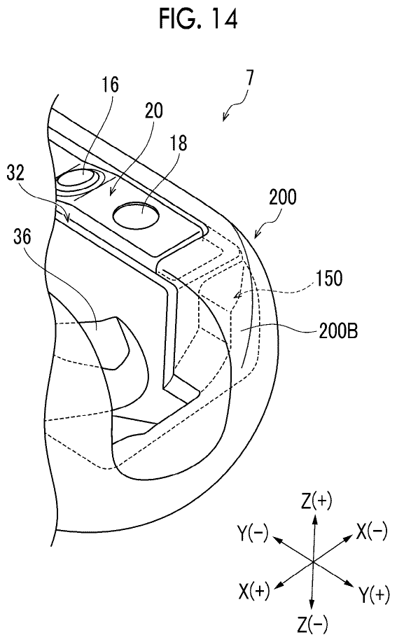

FIG. 14 is a perspective view showing main parts of the distal end portion 7 in which the distal end cap 200 is mounted on the distal end member 20. In FIG. 14, the protruding portion 150 is indicated by a broken line.

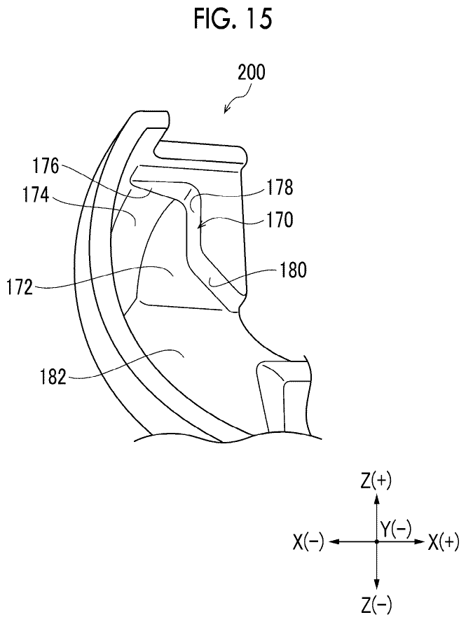

As shown in FIG. 14, in a case in which the distal end cap 200 is mounted on the distal end member 20, the protruding portion 150 is inserted into a mounting portion 170 (see FIG. 15) formed on the inner surface of the cap distal end portion 200B.

FIG. 15 is an explanatory view showing main parts of the mounting portion 170 of the distal end cap 200 as viewed from the Y(−) direction side. As shown in FIG. 15, the mounting portion 170 is formed in a recessed shape toward the Y(+) direction side on the inner surface of the distal end cap 200 on the Y(+) direction side. The mounting portion 170 has the following plurality of surfaces to engage with the protruding portion 150 inserted into the mounting portion 170.

That is, the mounting portion 170 has a flat surface 172 that abuts on (is in surface contact with; the same applies hereinafter) the distal end surface 152 of the protruding portion 150. This flat surface 172 has the same shape as the distal end surface 152. For example, in a case in which the distal end cap 200 is viewed through from the Y(+) direction side, the shape of the flat surface 172 viewed from the Y(+) direction and the shape of the distal end surface 152 viewed from the Y(+) direction are the same as each other.

Further, the mounting portion 170 has a curved surface 174 that abuts on the outer peripheral curved surface 154 of the protruding portion 150. This curved surface 174 has the same shape as the outer peripheral curved surface 154. For example, in a case in which the distal end cap 200 is viewed through from the Y(+) direction side, the shape of the curved surface 174 viewed from the Y(+) direction side and the shape of the outer peripheral curved surface 154 viewed from the Y(+) direction are the same as each other.

Further, the mounting portion 170 has a flat surface 176 that abuts on the upper surface 156 (second engaging surface) of the protruding portion 150. This flat surface 176 has the same shape as the upper surface 156. For example, in a case in which the distal end cap 200 is viewed through from the Z(+) direction side, the shape of the flat surface 176 viewed from the Z(+) direction and the shape of the upper surface 156 viewed from the Z(+) direction are the same as each other.

In addition, the mounting portion 170 has a flat surface 178 that abuts on the inner vertical surface 158 (first engaging surface). This flat surface 178 has the same shape as the inner vertical surface 158. For example, in a case in which the distal end cap 200 is viewed through from the X(+) direction side, the shape of the flat surface 178 viewed from the X(+) direction side and the shape of the inner vertical surface 158 viewed from the X(+) direction are the same as each other. It should be noted that the flat surface 178 and the flat surface 176 form an engaged portion according to the embodiment of the present invention.

Further, the mounting portion 170 has a slope 180 that abuts on the inner inclined surface 160. This slope 180 has the same shape as the inner inclined surface 160. For example, in a case in which the distal end cap 200 is viewed through from the X(+) direction side, the shape of the slope 180 viewed from the X(+) direction side and the shape of the inner inclined surface 160 viewed from the X(+) direction are the same as each other.

Further, the mounting portion 170 has an inclined surface 182 that abuts on the distal end inclined surface 162. This inclined surface 182 has the same shape as the distal end inclined surface 162. For example, in a case in which the distal end cap 200 is viewed through from the Y(+) direction side, the shape of the inclined surface 182 viewed from the Y(+) direction side and the shape of the distal end inclined surface 162 viewed from the Y(+) direction are the same as each other.

By inserting the protruding portion 150 into the mounting portion 170 having the plurality of surfaces, the protruding portion 150 engages with the mounting portion 170. It should be noted that the same shape here means a substantially same shape, and the shapes do not always mean that the shapes are completely the same as each other. That is, the shapes thereof are similar to each other as a whole, but a shape portion having a different shape may be included in a partial region. In addition, the shapes thereof are not limited to the same dimensions, and different dimensional portions may be included in a partial region.

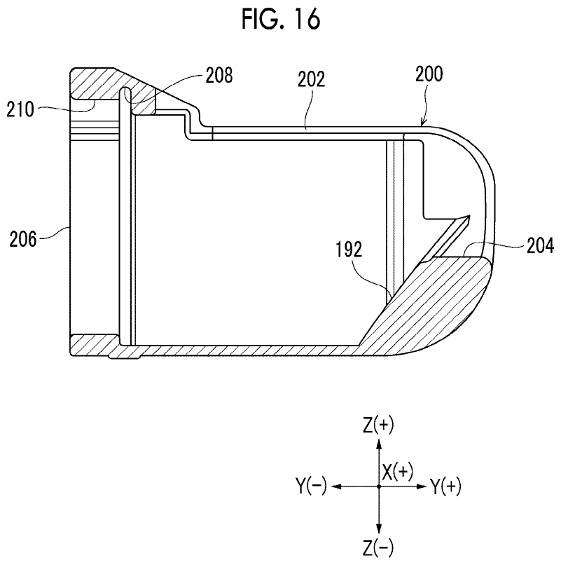

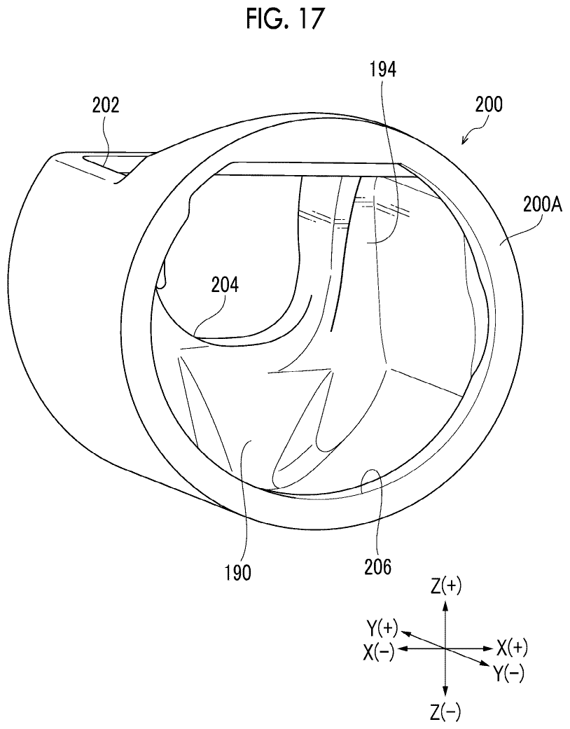

In addition, the distal end cap 200 includes an inclined surface 190 (see FIGS. 5 and 6), an inclined wall surface 192 (see FIG. 16), and an housing portion 194 (see FIG. 17), in addition to the mounting portion 170.

The inclined surface 190 shown in FIGS. 5 and 6 is provided on a surface on the Z(−) direction side of the inner peripheral surface of the distal end cap 200. The inclined surface 190 is a surface inclined to the Z(+) direction side from a substantially center position of the distal end cap 200 in the Y direction to a lower portion of the distal end portion opening 204. The inclined surface 190 functions as a surface that prevents foreign matter from being mixed in a gap between the elevator 36 and the inner surface of the distal end cap 200 in a case in which the distal end cap 200 is mounted on the distal end member 20.

The inclined wall surface 192 shown in FIG. 16 is provided on a surface on the Z(−) direction side of the inner peripheral surface of the distal end cap 200. The inclined wall surface 192 is provided on the X(−) side with respect to the inclined surface 190, and has a larger inclined angle of the inclined wall surface 192 than the inclined surface 190.

In addition, in a case in which the distal end cap 200 is mounted on the distal end member 20, the inclined wall surface 192 abuts on the wall surface abutment surface 165 (see FIG. 11) of the first wall portion 32. That is, the distal end cap 200 has the inclined wall surface 192 that can abut on the wall surface abutment surface 165.

FIG. 17 is a perspective view showing the distal end cap 200 as viewed from the Y(−) direction side. As shown in FIG. 17, the housing portion 194 is provided on the inner surface of the distal end portion of the distal end cap 200. The housing portion 194 is disposed on the X(+) side with respect to the distal end portion opening 204, and is formed as a recessed groove along the Z direction.

The housing portion 194 can house a part of the second wall portion 34, and specifically, houses the engaging portion 35 (see FIGS. 11, 12, and the like) provided on the Y(+) direction side of the second wall portion 34. The engaging portion 35 is formed as a thin-wall portion thinner than other portions on the Y(+) direction side. That is, the engaging portion 35 is formed in a plate shape thinner in the X direction than other portions of the second wall portion 34 except for the engaging portion 35. In a case in which the distal end cap 200 is mounted on the distal end member 20, the engaging portion 35 is inserted into the housing portion 194 and is housed therein. It should be noted that the housing portion 194 is an example of an housing portion according to the embodiment of the present invention.

### Effects of Endoscope 1

Hereinafter, the effects of the endoscope 1 according to the present embodiment will be described.

First, the distal end cap 200 is mounted on the distal end member 20 by externally mounting the distal end cap 200 on the distal end member 20 from the Y(+) side to the Y(−) side with the base end portion opening 206 of the distal end cap 200 as a leading end.

In this case, the engaging groove 208 of the distal end cap 200 is locked to the flange portion 30 of the distal end member 20, so that the distal end cap 200 is prevented from being disengaged from the distal end member 20. Further, since the flat portion 210 of the distal end cap 200 abuts on the flat portion 78 of the insulating ring 70, the rotation shift of the distal end cap 200 with respect to the distal end member 20 is prevented. Here, the flat portion 78 corresponds to a flat portion according to the embodiment of the present invention, which is disposed on the base end side (Y(−) direction side) with respect to the flange portion 30. In this case, the base portion according to the embodiment of the present invention is formed by the base portion 28 and the insulating ring 70.

In addition, in a case in which the distal end cap 200 is mounted on the distal end member 20, the protruding portion 150 of the distal end member 20 is inserted into the mounting portion 170 of the distal end cap 200, and the engaging portion 35 of the distal end member 20 is inserted into the housing portion 194 of the distal end cap 200. As a result, the distal end portion 7 is assembled.

In the distal end portion 7 having such a configuration, as described above, there is a case in which the cap distal end portion 200B of the distal end cap 200 is turned up from the distal end member 20 due to a force received from the body cavity wall during the treatment of the endoscope 1.

In particular, in a case in which a force is applied to the cap distal end portion 200B from the X(+) direction side to the X(−) direction side and in a case in which a force is applied from the Z(+) direction side to the Z(−) direction side, the cap distal end portion 200B is more likely to be turned up than in a case in which a force is applied from another direction.

Therefore, in the endoscope 1 according to the embodiment, for example, in a case in which a force is applied from the X(+) direction side to the X(−) direction side to the cap distal end portion 200B from the body cavity wall during the bending operation of the bendable portion 6, the force is received by the inner vertical surface 158 (first engaging surface) of the protruding portion 150 via the flat surface 178 of the mounting portion 170. Accordingly, since the deformation of the cap distal end portion 200B caused by the force in the above-described direction can be suppressed, the turning-up of the cap distal end portion 200B with respect to the distal end member 20 can be prevented.

In addition, for example, in a case in which a force is applied from the Z(+) direction side to the Z(−) direction side to the cap distal end portion 200B from the body cavity wall during the bending operation of the bendable portion 6, the force is received by the upper surface 156 (second engaging surface) of the protruding portion 150 via the flat surface 176 of the mounting portion 170. Accordingly, since the deformation of the cap distal end portion 200B caused by the force in the above-described direction can be suppressed, the turning-up of the cap distal end portion 200B with respect to the distal end member 20 can be prevented.

As described above, with the endoscope 1 according to the embodiment, the protruding portion 150 having the inner vertical surface 158 facing the X(+) direction side and the upper surface 156 facing the Z(+) direction side is provided on the end surface 110 of the first wall portion 32, and the engaged portion (flat surface 176 and flat surface 178) that engages with the inner vertical surface 158 and the upper surface 156 are provided on the distal end cap 200, so that it is possible to prevent the distal end cap 200 from being turned up during the treatment.

In addition, the distal end cap 200 can be mounted on the distal end member 20 only by inserting the protruding portion 150 into the mounting portion 170, and the distal end cap 200 can be removed from the distal end member 20 only by pulling out the protruding portion 150 from the mounting portion 170. Therefore, the attachment and detachment of the distal end cap 200 to and from the distal end member 20 are facilitated.

Therefore, with the endoscope 1 according to the present embodiment, it is possible to easily attach and detach the distal end cap 200 to and from the distal end member 20 and to eliminate the problem in which the distal end cap 200 is turned up during the treatment.

In addition, for example, in a case in which a force is applied from the Y(+) direction side to the Y(−) direction side to the cap distal end portion 200B from the body cavity wall in a case of the insertion operation of the insertion part 2, the force is received by the distal end surface 152 (see FIG. 13) of the protruding portion 150 via the flat surface 172 of the mounting portion 170. Accordingly, since the deformation of the cap distal end portion 200B caused by the force in the above-described direction can be suppressed, the turning-up of the cap distal end portion 200B with respect to the distal end member 20 can be prevented.

In addition, for example, in a case in which a force is applied from the X(−) direction side to the X(+) direction side to the cap distal end portion 200B from the body cavity wall in a case of the bending operation of the bendable portion 6, the force is received by the outer peripheral curved surface 154 of the protruding portion 150 via the curved surface 174 of the mounting portion 170. Accordingly, since the deformation of the cap distal end portion 200B caused by the force in the above-described direction can be suppressed, the turning-up of the cap distal end portion 200B with respect to the distal end member 20 can be prevented.

In addition, for example, in a case in which a force is applied from the Z(−) direction side to the cap distal end portion 200B from the body cavity wall during the bending operation of the bendable portion 6 toward the Z(+) direction side, the force is received by the distal end inclined surface 162 of the protruding portion 150 and the outer peripheral curved surface 154 via the flat surface 172 and the curved surface 174 of the mounting portion 170.

That is, the force in the above-described direction is received by two surfaces (distal end inclined surface 162 and outer peripheral curved surface 154) having a large area. Accordingly, since the deformation of the cap distal end portion 200B caused by the force in the above-described direction can be suppressed, the turning-up of the cap distal end portion 200B with respect to the distal end member 20 can be prevented.

### Other Effects

The distal end cap 200 is formed such that a wall thickness of the distal end portion of the distal end cap 200 is thicker than that of a case in which the slope 180 is not provided, in order to form the slope 180 that engages with the inner inclined surface 160 of the protruding portion 150.

That is, the strength of the distal end portion of the distal end cap 200 can be increased by forming the inner inclined surface 160 on the protruding portion 150 and forming the slope 180 that engages with the inner inclined surface 160 on the mounting portion 170.

In addition, by providing the inner inclined surface 160 on the protruding portion 150, the inner vertical surface 158 and the inner inclined surface 160 can be connected by an obtuse angle connection portion. As a result, the washability of the protruding portion 150 using a brush or the like can be improved as compared with a case in which the inner inclined surface 160 is not provided, that is, a case in which the end portion of the inner vertical surface 158 on the Z(−) direction side is angular (for example, a right angle).

In addition, since the distal end surface 152 of the protruding portion 150 and the distal end inclined surface 162 are connected to each other via the fillet surface 168, the washability of the protruding portion 150 using a brush or the like can be improved as compared with a case in which the distal end surface 152 and the distal end inclined surface 162 are connected to each other at a corner portion without the fillet surface 168.

Although the endoscope according to the embodiment has been described above, the present invention may be improved or modified in some ways without departing from the gist of the present invention.

### EXPLANATION OF REFERENCES

1: endoscope
2: insertion part
3: operating part
4: universal cord
5: soft portion
6: bendable portion
7: distal end portion
8: angle knob
9: angle knob
10: air/water supply button
11: suction button
12: elevating operation lever
13: treatment tool inlet port
14: air/water supply nozzle
16: observation window
18: illumination window
18A: disposition surface
20: distal end member
22: elevator housing portion
24: opening
26: distal end opening
28: base portion
28A: distal end wall portion
30: flange portion
30A: base end side surface
32: first wall portion
34: second wall portion
35: engaging portion
36: elevator
36A: guide surface
38: treatment tool insertion channel
39: treatment tool outlet port
40: cover
42: lever
42A: rotation shaft
44: lever housing chamber
46: operating wire
50: guide wire fixing portion
50A: distal end portion
70: insulating ring
72: flange portion
72A: distal end side surface
72B: base end side surface
74: cap mounting portion
74A: base end
76: bendable portion mounting portion
78: flat portion
80: arc portion
100: cap mounting surface
110: end surface
150: protruding portion
152: distal end surface
154: outer peripheral curved surface
154A: ridge line
156: upper surface
156A: ridge line
158: inner vertical surface
158A: ridge line
160: inner inclined surface
160A: ridge line
162: distal end inclined surface
162A: ridge line
164: surface
165: wall surface abutment surface
166: surface
168: fillet surface
170: mounting portion
172: flat surface
174: curved surface
176: flat surface
178: flat surface
180: slope
182: inclined surface
190: inclined surface
192: inclined wall surface
194: housing portion
200: distal end cap
200A: base end
200B: cap distal end portion
202: upper opening
204: distal end opening
206: base end portion opening
208: engaging groove
210: flat portion

What is claimed is:

1. An endoscope comprising:
a distal end member that is provided at a distal end portion of an insertion part; and
a distal end cap that is mounted on the distal end member,
wherein, in a case in which an axial direction of the distal end member is defined as a first direction, one direction of two directions orthogonal to the first direction and orthogonal to each other is defined as a second direction, and the other direction of the two directions is defined as a third direction,
the distal end member has an elevator housing portion in which an elevator is housed, and a first wall portion that is disposed in the elevator housing portion on one side in the second direction and that has an observation window and an illumination window,
the distal end cap has, on one side in the third direction, a cap opening portion that exposes the observation window, the illumination window, and the elevator housing portion,
the first wall portion has an engaging portion that is provided on an end surface on one side in the first direction,
the engaging portion has a first engaging surface that faces the other side opposite to the one side in the second direction, and a second engaging surface that faces the one side in the third direction, and
the distal end cap has an engaged portion that engages with the first engaging surface and the second engaging surface.

2. The endoscope according to claim 1,
wherein the engaging portion is a protruding portion that protrudes from the end surface to the one side in the first direction.

3. The endoscope according to claim 2,
wherein the protruding portion has an inner inclined surface that is connected to the first engaging surface and that is inclined toward the one side in the third direction with respect to the first engaging surface.

4. The endoscope according to claim 3,
wherein the protruding portion has
a distal end surface that is connected to the first engaging surface and the second engaging surface and that faces the one side in the first direction,
an outer peripheral curved surface that is connected to the second engaging surface and the distal end surface and that faces the one side in the second direction, and
a distal end inclined surface that is disposed on the other side in the third direction with respect to the distal end surface via a fillet surface and that is inclined toward both the one side in the first direction and the other side in the third direction.

5. The endoscope according to claim 2,
wherein the end surface of the first wall portion has, on a region on the other side in the third direction with respect to the protruding portion, an inclined wall surface that is inclined toward both the one side in the first direction and the other side in the third direction, and
the distal end cap has a wall surface abutment surface that is capable of abutting on the inclined wall surface in a case in which the distal end cap is mounted on the distal end member.

6. The endoscope according to claim 1,
wherein the first wall portion has a cap mounting surface that is disposed on the one side in the first direction with respect to the illumination window and that faces the one side in the third direction, and
the cap mounting surface is covered with the distal end cap in a case in which the distal end cap is mounted on the distal end member.

7. The endoscope according to claim 6,
wherein the cap mounting surface is disposed on the other side in the third direction with respect to a disposition surface of the illumination window.

8. The endoscope according to claim 6,
wherein the second engaging surface is disposed on the other side in the third direction with respect to the cap mounting surface.

9. The endoscope according to claim 1,
wherein the distal end member has a second wall portion that is disposed in the elevator housing portion on the other side in the second direction, and
the distal end cap has a housing portion that is capable of housing a part of the second wall portion.

10. The endoscope according to claim 9,
wherein the second wall portion has, on the one side in the first direction, a thin-wall portion that is formed to be thinner than other portions, and
the housing portion is capable of housing the thin-wall portion.

11. The endoscope according to claim 1,
wherein the distal end member has a base portion that is disposed in the first wall portion on the other side opposite to the one side in the first direction,
a flange portion that protrudes in a radial direction is provided in an outer peripheral portion of the base portion, and
the distal end cap has a cap locking portion that locks the flange portion.

12. The endoscope according to claim 11,
wherein the base portion has a flat portion that is disposed on the other side in the first direction with respect to the flange portion and of which a part in a circumferential direction is formed to be flat.
